(19) United States
(12) Patent Application Publication
(10) Pub. No.: US 2022/0076816 A1
(43) Pub. Date: Mar. 10, 2022

(54) WEARABLE MONITOR WITH MEMORY

(71) Applicant: Micron Technology, Inc., Boise, ID (US)

(72) Inventors: Yi Hu, Boise, ID (US); Brooke Spencer, Boise, ID (US); Deepti Verma, Boise, ID (US); Jennifer F. Huckaby, Boise, ID (US)

(21) Appl. No.: 17/012,998

(22) Filed: Sep. 4, 2020

(51) Int. Cl. G16H 40/63; G06N 20/00; A61B 5/0408

(52) U.S. Cl. CPC G16H 40/63; A61B 5/14532; A61B 5/04087; G06N 20/00

(57) ABSTRACT

Methods, devices, and systems related to a wearable monitor with memory are described. An example device may include an electrode integrated into a multi-chip package (MCP) memory device, the electrode to monitor health data of a wearer of the wearable monitor. The device can include a first processing resource coupled to the MCP memory device, the electrode, or both, to receive the monitored health data. The MCP memory device may store the received health data. The MCP memory device may also be coupled to a wireless communication device. The wireless communication device may transfer the stored health data to a computing device. The computing device may be communicatively coupled to the example device.

### TECHNICAL FIELD

The present disclosure relates generally to a device, and more particularly, to methods, apparatuses, and systems related to a wearable monitor with memory.

### BACKGROUND

Memory devices are typically provided as internal, semiconductor, integrated circuits in computers or other electronic devices. There are many different types of memory including volatile and non-volatile memory. Volatile memory can require power to maintain and process its data. Volatile memory may include random-access memory (RAM), dynamic random-access memory (DRAM), and synchronous dynamic random-access memory (SDRAM), among others. Non-volatile memory can provide persistent data by retaining stored data when not powered and may include NAND flash memory, NOR flash memory, read only memory (ROM), Electrically Erasable Programmable ROM (EEPROM), Erasable Programmable ROM (EPROM), and resistance variable memory such as phase change random-access memory (PCRAM), resistive random-access memory (RRAM), and magnetoresistive random-access memory (MRAM), among others.

### BRIEF DESCRIPTION OF THE DRAWINGS

FIG. 1 illustrates an example of a wearable monitor including a memory in accordance with a number of embodiments of the present disclosure.

FIG. 2 illustrates an example a wearable monitor with memory on a wearer in accordance with a number of embodiments of the present disclosure.

FIG. 3 illustrates another example of wearable monitor including a memory in accordance with a number of embodiments of the present disclosure.

FIG. 4 illustrates another example of a system including a wearable monitor and a computing device in accordance with a number of embodiments of the present disclosure.

FIG. 5 illustrates an example of a system including a wearable monitor in accordance with a number of embodiments of the present disclosure.

FIG. 6 is a flow diagram of a method for monitoring health data in accordance with a number of embodiments of the present disclosure.

### DETAILED DESCRIPTION

The present disclosure includes methods, apparatuses, and systems related to a wearable monitor with memory. An example device can include an electrode integrated into a multi-chip package (MCP) memory device, the electrode to monitor health data of a wearer of the wearable monitor. The wearable monitor can receive, at a processing resource coupled to the MCP memory device, the electrode, or both, the monitored health data, and the MCP memory device may store the received health data. The MCP memory device may also be coupled to a wireless communication device. The wireless communication device may transfer the stored health data to a computing device. The computing device may be communicatively coupled to the example device.

An example method can include receiving at a processing resource of a wearable monitor, health data of a wearer of the wearable monitor via an electrode of the wearable monitor integrated into an MCP memory device that is coupled to the processing resource, writing the received health data to the MCP memory device, and transferring the stored health data from the MCP memory device to a computing device using a wireless communication device communicatively coupled to the MCP memory device.

An example system can include a wearable monitor comprising an adhesive material attachable to human skin to secure the wearable monitor to a user, an electrode integrated into an MCP memory device to monitor health data, and a processing resource coupled to the MCP memory device, the electrode, or both, to receive the monitored health data. The MCP memory device can store the received health data, and non-transitory machine-readable instructions executable by the processing resource can transfer the stored health data to a computing device wirelessly via a wireless communication device.

An electrode may be used as a sensor to monitor a wearer's health data. The electrode may be used to measure electrical activity of a wearer's body and may detect electrical changes within the wearer's body. These electrical changes may be a consequence of cardiac muscle depolarization followed by repolarization during each cardiac cycle (e.g., heartbeat).

Some monitors may include multiple electrodes connected to a portable device to which the electrodes are connected to process the health data. The electrodes may have limited hydrophobic coating, and some electrodes may detect health indicators but may be unable to notify the wearer of health indicators.

In contrast, examples of the present disclosure can include the addition of memory to an electrode to monitor and process the wearer's health data. Some examples of the present disclosure can include integration of the electrode into an MCP memory device to increase speed and efficiency of an associated wearable monitor. Memory can be utilized as volatile and non-volatile data storage for a wide range of electronic applications. Non-volatile memory may be used in, for example, personal computers, portable memory sticks, digital cameras, cellular telephones, and other electronic devices. Memory cells may also be arranged into arrays to be used in memory devices. Memory modules may include volatile, such as DRAM, and/or non-volatile memory, such as Flash memory or RRAM, for example.

A wireless communication device, which may be referred to as a radio or modem, may also be added to the wearable monitor in some examples. The wireless communication device can provide for ease of upload and analysis of the wearer's health data. In one embodiment, the wearable monitor may be waterproof such that a wearer need not remove it during swimming, bathing, etc. The wireless communication device may be set to send notifications to a wearer or associated computing device when a health data threshold is reached. For example, the wireless communication device may send notifications when a monitored body function exceeds a threshold set by the wearer. The threshold may be a number such a temperature, heartbeats per second/minute, etc. The wireless communication device may send out notifications to the user, non-wearer, and/or a healthcare professional or an emergency personnel, based on user-based settings. For example, the wearer may be set to show and/or send a notification when the monitor records certain symptoms. Transmission of health data by the wireless communication device may be protected by encryption to secure the health data, by password, or both.

In the following detailed description of the present disclosure, reference is made to the accompanying drawings that form a part hereof, and in which is shown by way of illustration how one or more examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of the present disclosure. As used herein, “a number of” something can refer to one or more of such things. For example, a number of computing devices can refer to one or more computing devices. A “plurality” of something intends two or more.

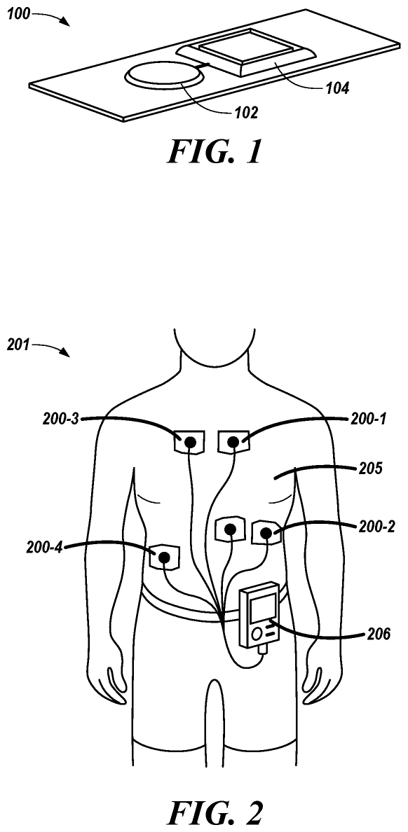

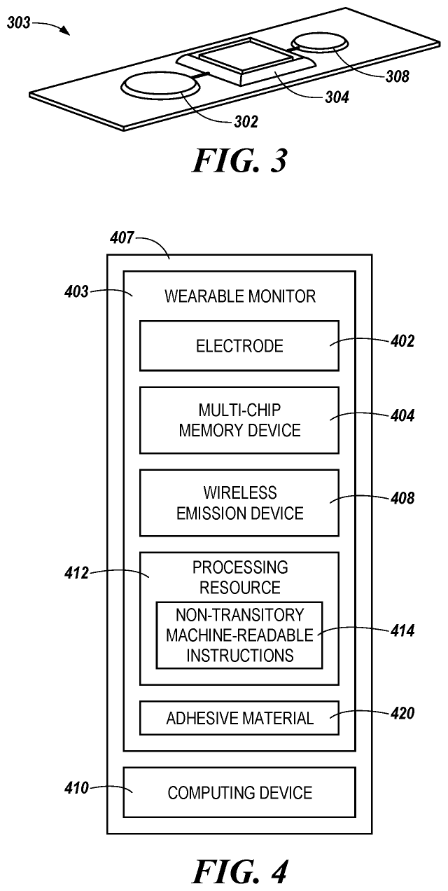

The figures herein follow a numbering convention in which the first digit or digits correspond to the drawing figure number and the remaining digits identify an element or component in the drawing. Similar elements or components between different figures may be identified by the use of similar digits. For example, reference numeral 102 may reference element “2” in FIG. 1, and a similar element may be referenced as 302 in FIG. 3. In some instances, a plurality of similar, but functionally and/or structurally distinguishable, elements or components in the same figure or in different figures may be referenced sequentially with the same element number (e.g., 200-1, 200-2, 200-3 in FIG. 2). As will be appreciated, elements shown in the various embodiments herein can be added, exchanged, and/or eliminated so as to provide a number of additional embodiments of the present disclosure. In addition, the proportion and the relative scale of the elements provided in the figures are intended to illustrate various embodiments of the present disclosure and are not to be used in a limiting sense.

FIG. 1 illustrates an example of a wearable monitor 100 including a memory 104 (e.g., an MCP memory device) in accordance with a number of embodiments of the present disclosure. As used herein a “wearable monitor” can refer to any electrical device which can be attached to the body to observe and record a health function of the body. Examples of a wearable monitor may include a blood sugar monitor, heartbeat monitor, pulse monitor, pacemaker, and/or redundant combinations thereof.

In a number of embodiments, the wearable monitor 100 may include an electrode 102 integrated into an MCP memory device 104. For instance, various parts or aspects of the MCP memory device 104 and the electrode 102 may be linked or coordinated such that the components function together as a system. In some examples, the electrode 102 may be communicatively coupled to the MCP memory device 104. As used herein, “communicatively coupled” can include coupled via various wired and/or wireless connections between devices for access to and/or for movement (transmission) of instructions (e.g., control signals, address signals, etc.) and data, as appropriate to the context. The coupling need not be a direct connection, and in some examples, can be an indirect connection. The electrode 102 may be used to monitor a wearer's health data. The health data may include human body functions. For example, the electrode 102 may be used to measure a body's electrical activity such as cardiac pulses and/or measure strength and timing of a body's electrical activity. The electrode may not conduct electricity into the body. In some examples, the electrode 102 may detect small electrical changes within the body, such as electrical changes that are a consequence of cardiac muscle depolarization followed by repolarization during each cardiac cycle (e.g., heartbeat). In some examples, the electrode 102 may be placed over bones to minimize interference from muscular activity.

In some examples, the wearable monitor 100 can include a processing resource (not illustrated in FIG. 1) coupled to the MCP memory device 104, the electrode 102, or both. The processing resource can receive the health data monitored by the electrode. For instance, the processing resource may process the health data to determine what is included in the health data (e.g., heartbeat activity) and assign metadata to the health data associated with contents of the monitored health data received at the processing resource. The processing resource, in some examples, may be implemented internally and on a same chip as at least a portion of the MCP memory device 104. For instance, at least a portion of the MCP memory device 104 can be configured as processor in memory (PIM) such that processing performance can be increased (e.g., decreased processing time and/or processing can happen in real time). A PIM can be a processor or other circuitry which is implemented internally and near to a memory (e.g., directly on a same chip as a memory array). The PIM can include hardware, firmware, and/or software to determine attributes of the incoming image data, generate rules, and select a memory media type to write the image data. A PIM may save time and/or power by reducing and/or eliminating external communications.

In some examples, the wearable device 100 comprises a system on a chip (SoC). For instance, a portion of the wearable device 100, a portion of the MCP memory device 104, or both can include a chip that integrates a plurality of components (e.g., the MCP memory device 104, a processor, the electrode 102, secondary storage, etc.).

In some examples, the electrode 102 of the wearable monitor may be used as a sensor to take a measurement of the wearer's health data. The electrode 102 may subsequently transfer the information collected during the measurement to the processing resource to process the health data as it is received, and the MCP memory device 104, as discussed further herein, may store the health data. In some examples (e.g., PIM, SoC, etc.) processing of the health data may occur at, near, or on the MCP memory device 104.

As noted, the wearable monitor 100 may also include the MCP memory device 104. The MCP memory device 104 may include a number of memory dies and/or chips each having one or more memory units. The dies may be, for example, NAND dies including a number of arrays of NAND flash memory cells and associated peripheral circuitry (e.g., write circuitry, read circuitry, I/O circuitry, buffers, etc.). The dies may also be DRAM cells. The MCP memory device 104 may include a unit of volatile memory and may also include a unit of non-volatile memory, among other types of memory. That is, the MCP memory device 104 may include a unit of NAND flash memory and also a unit of DRAM memory. As such, the MCP memory device 104 may be capable of both storing and processing health data in real time. As used herein, “real time” may be the current time the health data is being sent to the MCP memory device 104. That is, the health data may be stored and processed such that there is no noticeable delay experienced by the wearer. For example, the MCP memory device 104 may store the received heath data as it is monitored by the electrode 102, received by the processing resource, or both. In some examples, the health data may be stored locally within the MCP memory device 104. For instance, local storage can allow for fast retrieval of the health data, increasing efficiency and reporting times of the wearable device 100.

Data within the MCP memory device 104, transferred to the MCP memory device 104, and/or transferred from the MCP memory device 104 may be encrypted for security of the wearer's information. As used herein, “encryption” is the process of encoding a message or information in such a way that only authorized parties can access it. The purpose of data encryption is to protect digital data confidentiality as it is stored on computer systems and transmitted using the internet or other computer networks. The encryption translates the health data into code, so that only authorized people with access to a decryption key can read it. The encryption may protect the device itself. That is, access to the MCP memory device 104 may be limited by the encryption. The wearer may have the key to decrypt the information. The wearer may give the key to a healthcare professional and anyone identified by the wearer.

In some examples, the wearable monitor 100 can include non-transitory machine-readable instructions (not illustrated in FIG. 1) stored in the MCP memory device 104 or other storage that are executable by the processing resource to transfer the stored health data from the MCP memory device 104 to a different processing resource communicatively coupled to the MCP memory device 104. For instance, a second processing resource (not illustrated in FIG. 1), may be used to further process the health data.

In some examples, the wearable monitor 100 may be attachable to human skin by an adhesive material. As such, the wearable monitor 100 may be placed on or within the body of the user. In some examples, the wearable monitor 100 may be waterproof such that the wearer may wear it at all times without needing to take it off (e.g., during bathing, swimming, etc.).

FIG. 2 illustrates an example of a wearable monitor with memory on a wearer in accordance with a number of embodiments of the present disclosure. The example view 201 and associated wearable monitors may include the same or similar elements as the wearable monitor 100 as referenced in FIG. 1. For example, the wearable monitors 200-1, 200-2, 200-3, 200-4 (collectively referred to herein as wearable monitors 200) may be analogous or similar to the wearable monitor 100.

In a number of embodiments, a plurality of wearable monitors 200 may be used. The wearable monitors 200 may be placed at intervals on the wearer's body 205, as illustrated by the different locations of wearable monitors 200-1, 200-2, 200-3, 200-4. While four wearable monitors 200 are illustrated in FIG. 2, more or fewer wearable monitors 200 may be present. A plurality of wearable monitors 200 may be used when precise ECG signal information is desired to analyze the nature and origin of the measurement received by the electrode (as shown as 102 in FIG. 2).

The wearable monitors 200 (e.g., processing resources associated therewith, MCP memory devices associated therewith, etc.) may be communicatively coupled to a computing device 206 using wire. As used herein a “computing device” can refer to any electrical device which runs using an operating system that is loaded onto the electronic device. Examples of a computing device may include a personal laptop computer, a desktop computer, a mobile phone, a smart phone, a tablet, a wrist-worn device, a digital camera, and/or redundant combinations thereof. Although the computing device 206 is illustrated as a portable device continuously connected to the wearable monitors 200, embodiments of the present disclosure are not so limited.

The computing device 206 may be accessible by a healthcare professional to read health data stored within the MCP memory device (as shown by 104 in FIG. 1). The computing device 206 may also be a device accessible by the wearer 205 to transfer the health data stored within the MCP memory device to a healthcare professional or monitor his or her health data.

In some examples, due to the ability of the wearable monitors 200 to store and process the health data as received, the wearer 205 may not wear or connect the computing device 206 until the health data is requested by a healthcare professional for analysis. That is, the wearer 205 may attach the wearable monitors 200 to the computing device 206 to give access to the healthcare professional to the health data on and as-requested basis. For example, health data can be stored locally on an MCP memory device of the wearable monitors 200 and transferred to the computing device 206 as desired.

The stored health data within the MCP memory device of the wearable monitors 200 may be accessed by using a password. As used herein, the term password refers to a form of security used for user authentication to gain access to a system. For example, it may include, but is not limited to a pin number, a passcode, a passkey, an eye scan, facial recognition, etc. That is, a password may be required to access and transfer the health data within the wearable monitors 200 to the computing device 206.

FIG. 3 illustrates another example of a wearable monitor 303 including a memory 304 in accordance with a number of embodiments. The wearable monitor 303 may include the same or similar elements as the wearable monitor 100 and/or wearable monitors 200 as referenced in FIGS. 1 and 2, respectively. For example, the electrode 102 may be analogous or similar to electrode 302. The MCP memory device 104 may be analogous or similar to MCP memory device 304.

A wireless communication device 308 may be communicatively coupled to the MCP memory device 304, which may be communicatively coupled to the electrode 302. In some examples, the wireless communication device 308 may be communicatively coupled to a processing resource (not illustrated in FIG. 3). The processing resource may be communicatively coupled to the MCP memory device 304, the electrode 302, or both. Although the wireless communication device 308 is illustrated as being communicatively coupled to the MCP memory device 304, embodiments of the present disclosure are not so limited. The wireless communication device 308 may be communicatively coupled to the electrode 302 which may be communicatively coupled to the MCP memory device 304. Embodiments having a location of the wireless communication device 308 not specified herein may also be permissible. The wireless communication device 308, in some examples, may be coupled by a wired connection, a wireless connection, or a combination thereof to the MCP memory device 304, a processor, the electrode 302, or a combination thereof.

The wireless communication device 308 may transmit the health data stored within the MCP memory device 304 to a computing device (not illustrated in FIG. 3). The wireless communication device 308 may be coupled by a wired connection or a wireless connection to the computing device. The wearer (shown as 205 in FIG. 2) may determine when to transfer the health data from the wearable monitor 303 to the computing device. The wearer may set automated times at which the health data should be uploaded to the computing device. In one example, the wearer may set automated times at which the stored health data may be uploaded directly to a data server of a healthcare professional. In another example, the wearer may transfer the stored health data to an Artificial Intelligence (AI) for analysis of the health data.

The transfers may occur in real time, as the health data is received and processed by a processing resource and/or the MCP memory device 304 of the wearable monitor 303. The wearer may also determine when to transfer the health data to the computing device. The wearer may send a notification along with health data. For example, the wearer may send a notification to an emergency personnel about certain health symptoms processed by the processing resource and/or the MCP memory device 304 of the wearable monitor 303.

In some embodiments, a wearer may notify a healthcare professional at certain intervals to signal a particular health indicator responsive to communication from the wearable monitor 303. For example, a wearer may be advised to signal the healthcare professional when the body experiences an abnormal health event. The abnormal health event may range from a racing heart to a fatal heart attack symptom, among others. The wearable monitor 303 may wirelessly send a notification to the wearer who may then approve for the stored health data and the notification to be sent to the health professional. The wearer may send the notification with the stored health data from the MCP memory device 304 to signal a healthcare professional of the abnormal event. In some examples, approval from a wearer may not be obtained, for instance when the abnormal event is serious, such as a heart attack.

The wearable monitor 303 may also send an alarm notification to the wearer when a potentially fatal abnormal event is occurring. As used herein, “potentially fatal abnormal event” may refer to a health occurrence that may be indicative of a serious health issue. For example, it may include but is not limited to, heart attacks, shortness of breath, stroke, paralysis, or other serious health occurrences.

The wearer may set what the threshold for a potentially fatal abnormal event. A healthcare professional may also set the threshold for a potentially fatal abnormal event. The threshold set by the healthcare professional may be changed by the wearer. The wearer may also set automatic notifications to be sent to emergency personnel during the potentially fatal abnormal event. That is, a notification may be sent by the wireless communication device 308 of the wearable monitor 303 to an emergency personnel based on detection of a potentially fatal abnormal event.

As used herein, “alarm notification” may refer to a signal to bring attention to information. For example, it may include, but is not limited to vibration, sound/voiced signal, text message, and phone call. The length and duration of the alarm notification may be set by either the healthcare professional or the wearer.

Access to the stored health data within the MCP memory device 304 of the wearable monitor 303 may be received using a password. That is, a password may be required to access and transfer the health data within the MCP memory device 304 of the wearable monitor 303 to the computing device (illustrated as 206 in FIG. 2). The wearer may adjust settings such that the transfer of health data to the healthcare professional does not require a password.

FIG. 4 illustrates another example of a system 407 including a wearable monitor 403 and a computing device 410 in accordance with a number of embodiments of the present disclosure. The wearable monitor 403 includes an electrode 402, a wireless communication device 408, an MCP memory device 404, an adhesive material 420, a processing resource 412 coupled to the MCP memory device 404, the electrode 402, or both, and non-transitory machine-readable instructions 414 executable by the processing resource 414.

The adhesive material 420 can include a material that is attachable to human skin to secure the wearable monitor 403. In some instances, the wearable monitor 403 may be adhered within the wearer's body. The electrode 402 may be integrated into the MCP memory device 404 to monitor a wearer's health data. For instance, the health data may include human body functions, and the electrode 402 may be used as a sensor to take measurements of the body's internal functions. Examples include the strength and timing of a body's electrical activity such as cardiac pulses. In some examples, the electrode 402 may be communicatively coupled to the MCP memory device 404, the processing resource, or both. The electrode 402 may monitor the health data, and the processing resource 412 may retrieve the monitored health data and transfer it to the MCP memory device 404 for storage.

The processing resource 412 can be execute the instructions 414 to transfer the stored health data to a computing device 410 wirelessly via a wireless communication device. For instance, the processing resource 412 may retrieve the stored health data from the MCP memory device 404 and transfer it to a processing resource of the computing device 410. In some examples, the processing resource 412 can simultaneously transfer the received health data to a data server and send a notification to the wearer. For example, the processing resource 412 may upload the wearer's health data from the wearable monitor 403 to a data server and at same time, send notification about that same health data to the wearer. The wireless communication device may be used to send the notification responsive to receiving the wearer's approval to send the notification.

The processing resource 412, in some examples, can be implemented internally and on a same chip as at least a portion of the MCP memory device 404 to process the received health data in real time. This can increase efficiency in data transfer by decreasing transfer time and increasing processing speed, in some examples.

In some examples, the wireless communication device 408 may transmit the health data stored within the MCP memory device 404 to the computing device 410, and the computing device 410 may receive data from the wireless communication device 408. The wearer may determine when to transfer the health data from the wearable monitor 403 to the computing device 410. For example, the wearer may set automated times at which the health data should be uploaded to the computing device 410. In one example, the wearer may set automated times at which the stored health data may be uploaded directly to a data server of a healthcare professional. The transfers may occur in real time, as the health data is received and processed by the processing resource 412 and/or the MCP memory device 404 of the wearable monitor 403. The wearer may also determine when to transfer the health data to the computing device. The wearer may send a notification about the health data to a wearer or associated computing device along with health data.

The computing device 410 may be accessible by a healthcare professional to read health data uploaded by the MCP memory device 404. The computing device 410 may also be a device accessible by the wearer to transfer the health data stored within the MCP memory device to a healthcare professional.

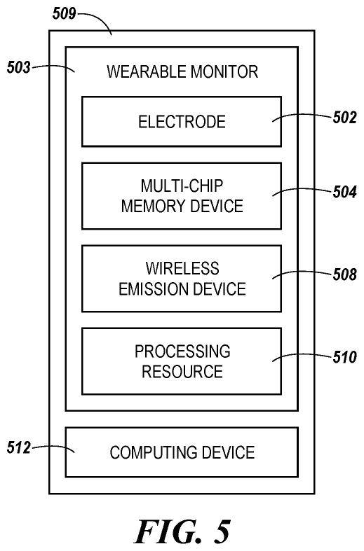

FIG. 5 illustrates an example of a system 509 including a wearable monitor 503 in accordance with a number of embodiments of the present disclosure. The system 509 may include the wearable monitor 503, a processing resource 512, and a computing device 510. The wearable monitor 503 includes an electrode 502, an MCP memory device 504, and a wireless communication device 508. In some examples, the processing resource 512 may be located on the wearable monitor 503.

The electrode 502 may be used to monitor a wearer's health data such as the wearer's body functions. The electrode 502 may be communicatively coupled to the MCP memory device 504, the processing resource 512, or both, and the MCP memory device 504 may be communicatively coupled to the electrode 502, the processing resource 512, or both. The processing resource 512 can retrieve or receive health data from the electrode 502 and may transfer the health data to the MCP memory device 504 for storage.

Health data stored within the MCP memory device 504 may be encrypted for security of the wearer's information. The encryption translates the health data into code, so that only authorized people with access to a decryption key can read it. The encryption may protect the device itself. That is, access to the MCP memory device 504 may be limited by the encryption.

The MCP memory device may include a processing in memory (PIM) memory. The MCP memory device may also include a System on a Chip (SoC) memory system. As such, the MCP memory device may be capable of both storing and processing health data in real time.

The processing resource 512, which may exist outside the MCP memory device 504, may be communicatively coupled to the MCP memory device 504, the electrode 502, the wireless communication device 508, the computing device 510, or a combination thereof. The processing resource 512 may be configured to execute executable instructions stored in MCP memory device 504 to upload the wearer's health data to the computing device 510 or a separate server. The processing resource 512 may transfer the health data using the via a wireless communication device 508. In some examples, more than one processing resource is present on the system 509.

The processing resource 512 may be configured to execute executable instructions stored in the MCP memory device 504 to transfer the received health data in real time via the wireless communication device 508. The wireless communication device 508 may transmit the health data stored within the MCP memory device 504 to the computing device 510. The wearer may determine when to transfer the health data from the wearable monitor 503 to the computing device 510. The computing device 510 (e.g., a processing resource thereon) may receive data from the wireless communication device 508.

The transfers may occur in real time, as the health data is received and processed by the MCP memory device 504 of the wearable monitor 503. The wearer may also determine when to transfer the health data to the computing device. The wearer may send a notification along with health data.

The computing device 510 may be accessible by a healthcare professional to read health data uploaded by the MCP memory device 504. The computing device 510 may also be a device accessible by the wearer to transfer the health data stored within the MCP memory device to a healthcare professional.

The processing resource 512 may be configured to execute executable instructions stored in the MCP memory device 504 to notify a healthcare professional or associated computing device at certain intervals to signal a particular health indicator. For example, a wearer may be advised to signal the healthcare professional when the body experiences an abnormal health event. The abnormality may be as mild as a racing heart or as fatal as heart attack symptoms. The processing resource 512 may also be configured to execute executable instructions stored in the MCP memory device 504 to send a notification to the wearer who may then approve for the stored health data and the notification to be sent to the health professional. The notification may appear on the computing device 510.

The computing device 510 includes a user interface. The wearer of the MCP memory device 504 (e.g., a user) of computing device 510, may interact with computing device 510 via a user interface shown on a display. For example, the user interface can provide (e.g., display and/or present) information to the user of computing device 510, and/or receive information from (e.g., input/selection by) the user of computing device 510. The display showing the user interface may be, for instance, a touchscreen (e.g., the computing device 510 can include touchscreen capabilities).

The wearer may send the notification with the stored health data from the MCP memory device 504 to signal a healthcare professional of the abnormal event. The processing resource 512 may also send an alarm notification to the wearer when a potentially fatal abnormal event is occurring. The wearer may set what the threshold for a potentially fatal abnormal event. A healthcare professional may also set the threshold for a potentially fatal abnormal event. The threshold set by the healthcare professional may be changed by the wearer. The wearer may also be able to choose a setting in the wearable monitor to send automated notification to emergency personnel during the potentially fatal abnormal event. That is, a notification may be sent by the processing resource 512, via the wireless communication device 508 of the wearable monitor 503 to an emergency personnel based on detection of a potentially fatal abnormal event.

In another example, the processing resource 512 may be configured to execute executable instructions stored in the MCP memory device 504 to send the stored health data to an AI device for analysis of the health data. The AI may interpret the health data and inform the wearer and/or a healthcare professional that a potentially fatal abnormal event is occurring. The AI may also compare the health data received by the computing device 510 with health data received by patients with similar health concerns. The AI may highlight trends in health patterns and recommend treatment plans.

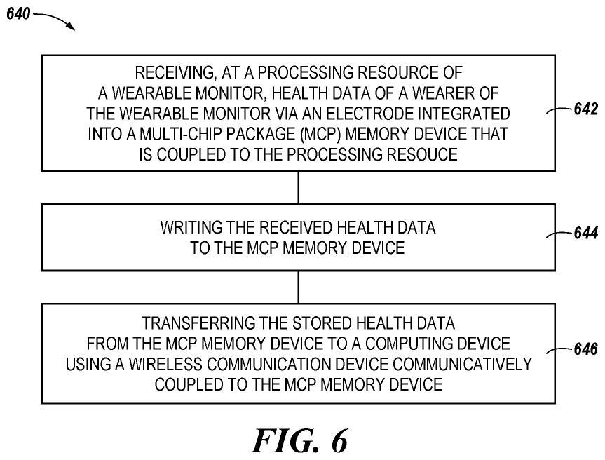

FIG. 6 is a flow diagram of a method 640 for monitoring health data in accordance with a number of embodiments of the present disclosure. At block 642, the method 640 may include receiving, at a processing resource of a wearable monitor, health data of a wearer of the wearable monitor via an electrode integrated into an MCP memory device that is coupled to the processing resource. The wearable monitor, for instance, may be analogous to wearable monitor 303, 403 and 503 as described in FIGS. 3-5. The wearable monitor may be analogous to wearable monitor 100 and 200 as described in FIGS. 1-2 if the wearable monitor is communicatively coupled to a computing device by wire. In a number of embodiments, the electrode may monitor a wearer's health data such as human body functions. For instance, the processing resource can receive health data of the wearer's heart functions via the electrode.

In some examples, the processing resource can send notifications, including alarm notifications, associated with the received health data to the computing device using the wireless communication device responsive to detection of a threshold health indicator by the electrode. For instance, if the electrode detects a reduced heartbeat (e.g., below a threshold beats-per-minute), the processing resource can send a notification. In such examples, the processing resource may send a notification to emergency personnel. For instances, a wearer may program the wearable monitor to automatically alert emergency personnel in particular situations. Automatically, as used herein, can include without human interaction and/or prompting.

At block 644, the method 640 may include a processing resource writing the received health data to the MCP memory device. In some examples, the processing resource may retrieve from the electrode the health data and transfer it to the MCP memory device for storage. The MCP memory device may also include an SoC memory system and/or a PIM device in some examples. For example, the method 640 can include receiving the health data at the processing resource, wherein the processing resource is implemented internally and on a same chip as at least a portion of the MCP memory device and processing, by the processing resource, the received health data in real time as the health data is received at the processing resource. In such examples, the MCP memory device may be capable of both storing and processing health data in real time. As used herein, “real time” may be the current time the health data is being sent to the MCP memory device. That is, the health data may be stored and processed such that there is no noticeable delay experienced by the wearer. In some examples, the processing resource can restrict access to the stored health data using a password, encryption, or both.

At block 646, the method 640 may include transferring the stored health data from the MCP memory device to a computing device using a wireless communication device communicatively coupled to the MCP memory device. For instance, a processing resource communicatively coupled to the MCP memory device may retrieve the health data from the MCP memory device and transmit the health data via the wireless communication device to which the processing resource is communicatively coupled. In some examples, the wireless communication device may be communicatively coupled to the MCP memory device, which may be communicatively coupled to the electrode. The wireless communication device may also be communicatively coupled to the electrode which may be communicatively coupled to the MCP memory device. Embodiments having a location of the wireless communication device not specified herein may be permissible.

In the above detailed description of the present disclosure, reference is made to the accompanying drawings that form a part hereof, and in which is shown by way of illustration how one or more examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of the present disclosure.

It is to be understood that the terminology used herein is for the purpose of describing particular examples only and is not intended to be limiting. As used herein, the singular forms “a”, “an”, and “the” include singular and plural referents, unless the context clearly dictates otherwise, as do “a number of”, “at least one”, and “one or more” (e.g., a number of memory arrays may refer to one or more memory arrays), whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to”. The terms “coupled” and “coupling” mean to be directly or indirectly connected physically and, unless stated otherwise, can include a wireless connection for access to and/or for movement (transmission) of instructions (e.g., control signals, address signals, etc.) and data, as appropriate to the context.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A wearable monitor comprising:
an electrode integrated into a multi-chip package (MCP) memory device, the electrode to monitor health data of a wearer of the wearable monitor;
a first processing resource coupled to the MCP memory device, the electrode, or both, to receive the monitored health data; and
the MCP memory device to store the monitor health data.

2. The wearable monitor of claim 1, wherein at the first processing resource is implemented internally and on a same chip as at least a portion of the MCP memory device.

3. The wearable monitor of claim 1, wherein the received health data is stored locally within the MCP memory device.

4. The wearable monitor of claim 1, wherein the wearable monitor comprises a system on a chip (SoC).

5. The wearable monitor of claim 1, further comprising:
the first processing resource coupled to the MCP memory device to access the stored health data; and
non-transitory machine-readable instructions stored in the MCP memory device and executable by the first processing resource to:
transfer the stored health data from the MCP memory device to a second processing resource communicatively coupled to the MCP memory device.

6. The wearable monitor of claim 1, wherein the MCP memory device stores the received health data in real time as the health data is monitored by the electrode, received by the first processing resource, or both.

7. A method for monitoring health data comprising:
receiving, at a processing resource of a wearable monitor, health data of a wearer of the wearable monitor via an electrode of the wearable monitor integrated into a multi-chip package (MCP) memory device that is coupled to the processing resource;
writing the received health data to the MCP memory device; and
transferring the stored health data from the MCP memory device to a computing device using a wireless communication device communicatively coupled to the MCP memory device.

8. The method of claim 7, further comprising receiving at the processing resource health data of the wearer's heart functions via the electrode.

9. The method of claim 7, further comprising the processing resource sending notifications associated with the received health data to the computing device using the wireless communication device responsive to detection of a threshold health indicator by the electrode.

10. The method of claim 9, further comprising the processing resource sending the notifications to emergency personnel responsive to detection of the threshold health indicator by the electrode.

11. The method of claim 7, further comprising:
receiving the health data at the processing resource, wherein the processing resource is implemented internally and on a same chip as at least a portion of the MCP memory device; and
processing, by the processing resource, the received health data in real time as the health data is received at the processing resource.

12. The method of claim 7, further comprising the processing resource restricting access to the stored health data using a password.

13. The method of claim 7, further comprising the processing resource providing an alarm notification to a wearer of the wearable monitor responsive to detection of a threshold health indicator by the electrode.

14. A system for monitoring health data, comprising:
a computing device; and
a wearable monitor comprising:
an adhesive material attachable to human skin to secure the wearable monitor to a wearer;
an electrode integrated into a multi-chip package (MCP) memory device to monitor health data;
a processing resource coupled to the MCP memory device, the electrode, or both, to receive the monitored health data;
the MCP memory device to store received the health data; and
non-transitory machine-readable instructions executable by the processing resource to transfer the stored health data to the computing device wirelessly via a wireless communication device.

15. The system of claim 14, further comprising the processing resource to simultaneously transfer the received health data to a data server and send a notification to the wearer.

16. The system of claim 15, further comprising the wireless communication device to send the notification responsive to receiving, via the computing device, wearer approval to send the notification.

17. The system of claim 14, further wherein the processing resource is implemented internally and on a same chip as at least a portion of the MCP memory device to process the received health data in real time.

18. The system of claim 14, wherein the wireless communication device sends the stored health data to an Artificial Intelligence (AI) device for analysis.

19. The system of claim 14, wherein the health data stored within the MCP memory device is encrypted.

20. The system of claim 14, wherein the wearable monitor is waterproof.
